(19) United States
(12) Patent Application Publication
(10) Pub. No.: US 2025/0014238 A1
(43) Pub. Date: Jan. 9, 2025

(54) SYSTEMS AND METHODS FOR MEDICAL IMAGE RECONSTRUCTION

(71) Applicant: SHANGHAI UNITED IMAGING HEALTHCARE CO., LTD., Shanghai (CN)

(72) Inventors: Jing YAN, Shanghai (CN); Juan FENG, Shanghai (CN)

(73) Assignee: SHANGHAI UNITED IMAGING HEALTHCARE CO., LTD., Shanghai (CN)

(21) Appl. No.: 18/893,937

(22) Filed: Sep. 23, 2024

Related U.S. Application Data
(63) Continuation of application No. PCT/CN2023/085765, filed on Mar. 31, 2023.

(30) Foreign Application Priority Data
Mar. 31, 2022 (CN) 202210332081.X

(51) Int. Cl. G06T 11/00

(52) U.S. Cl. CPC G06T 11/003; G06T 2207/20081

(57) ABSTRACT

Systems and methods for medical image reconstruction are provided. The systems may determine a target temporal resolution of a target scan on a target subject within a scanning angle range. The system may determine, based on the target temporal resolution, a plurality of reconstruction angle ranges each of which being within the scanning angle range. The system may obtain scan data acquired by causing an imaging device to perform the target scan on the target subject within the scanning angle range. The system may generate a plurality of reconstruction images corresponding to the plurality of reconstruction angle ranges based on the scan data.

### CROSS-REFERENCE TO RELATED APPLICATIONS

This application is a continuation of International Application No. PCT/CN2023/085765, filed on Mar. 31, 2023, which claims priority of Chinese Patent Application No. 202210332081.X, filed on Mar. 31, 2022, the contents of which are hereby incorporated by reference.

### TECHNICAL FIELD

The present disclosure generally relates to medical technology, and more particularly relates to systems and methods for medical image reconstruction.

### BACKGROUND

Perfusion imaging can reflect the blood flow microcirculation of a contrast agent in tissues or lesions, which is of great help in differentiating benign and malignant tumors and understanding the blood supply of cerebral ischemic lesions. Therefore, perfusion imaging is widely used in the diagnosis of various diseases.

Perfusion imaging often needs to obtain the reconstruction results of the contrast agent in the whole scanning process (e.g., from the moment when the contrast agent enters the tissue or the lesion to the moment when the contrast agent leaves the tissue or the lesion), and the scanning process is short. To obtain accurate reconstruction results, existing methods need to inject a contrast agent multiple times and perform multiple perfusion scans. However, multiple injections of the contrast agent may cause some damage to the patient, and the scanning efficiency is low due to the long scanning time. Therefore, it is desired to provide systems and methods for perfusion imaging and image reconstruction that can reduce the number of injections of the contrast agent while obtaining accurate reconstruction results.

### SUMMARY

In one aspect of the preset disclosure, a system for medical image reconstruction is provided. The system may include at least one storage device including a set of instructions and at least one processor configured to communicate with the at least one storage device. When executing the set of instructions, the at least one processor may be configured to direct the system to perform following operations including, determining a target temporal resolution of a target scan on a target subject within a scanning angle range, determining, based on the target temporal resolution, a plurality of reconstruction angle ranges each of which being within the scanning angle range, obtaining scan data acquired by causing an imaging device to perform the target scan on the target subject within the scanning angle range, and generating a plurality of reconstruction images corresponding to the plurality of reconstruction angle ranges based on the scan data.

In some embodiments, at least one overlapping angle range may exist between at least one pair of adjacent reconstruction angle ranges of the plurality of reconstruction angle ranges, one overlapping angle range corresponding to one of the at least one pair of adjacent reconstruction angle ranges.

In some embodiments, the at least one overlapping angel range may include a first overlapping angel range between a first pair of adjacent reconstruction angle ranges and a second overlapping angel range between a second pair of adjacent reconstruction angle ranges. A span of the first overlapping angel range may be different from a span of the second overlapping angel range.

In some embodiments, the obtaining scan data may include obtaining real-time scan data collected by the imaging device during the target scan. The generating a plurality of reconstruction images corresponding to the plurality of reconstruction angle ranges based on the scan data may include for each of the plurality of reconstruction images, starting image reconstruction based on the real-time scan data after the imaging device rotates to a starting angle of the corresponding reconstruction angle range.

In some embodiments, the target scan may be a perfusion scan. The determining a target temporal resolution may include obtaining reference information relating to a contrast agent used in the perfusion scan and determining the target temporal resolution based on the reference information. The reference information may include at least one of a contrast agent concentration of the contrast agent, an injection volume, an injection speed, a development stage of the contrast agent, or accuracy requirement of perfusion parameters.

In some embodiments, the determining a plurality of reconstruction angle ranges may include obtaining a rotation speed of the imaging device during the target scan, determining a reference value of a step based on the rotation speed and the target temporal resolution, the step being an angle interval between adjacent reconstruction angle ranges among the plurality of reconstruction angle ranges, and determining the plurality of reconstruction angle ranges based on the reference value.

In some embodiments, the reference value of the step may include a plurality of reference values, and the angle interval between adjacent reconstruction angle ranges among the plurality of reconstruction angle ranges may be changing.

In some embodiments, the target scan may be a perfusion scan. In some embodiments, the target temporal resolution may include a plurality of target temporal resolutions corresponding to a plurality of development stages of a contrast agent used in the perfusion scan, and each of the plurality of reference values may be determined based on one of the plurality of target temporal resolutions and the rotation speed.

In some embodiments, the at least one processor may be configured to direct the system to perform operations including generating one or more predicted reconstruction images based on the plurality of reconstruction images. Each of the one or more predicted reconstruction images may correspond to a reconstruction angle range that is between two adjacent reconstruction angle ranges among the plurality of reconstruction angle ranges.

In some embodiments, the generating the one or more predicted reconstruction images based on the plurality of reconstruction images may include obtaining a first trained image prediction model and generating the one or more predicted reconstruction images by inputting the plurality of reconstruction images into the first trained image prediction model.

In some embodiments, the generating the one or more predicted reconstruction images based on the plurality of reconstruction images may include obtaining at least one pair of adjacent reconstruction images among the plurality of reconstruction images, obtaining a second trained image prediction model, and for each of the at least one pair of adjacent reconstruction images, generating a predicted reconstruction image by inputting the pair of adjacent reconstruction images into the second trained image prediction model.

In some embodiments, a temporal resolution of the plurality of reconstruction images may be equal to or higher than the target temporal resolution.

In another aspect of the present disclosure, a method for medical image reconstruction is provided. The method may be implemented on a computing device including at least one processor and at least one storage device. The method may include determining a target temporal resolution of a target scan on a target subject within a scanning angle range. The method may include determining, based on the target temporal resolution, a plurality of reconstruction angle ranges each of which being within the scanning angle range. The method may include obtaining scan data acquired by causing an imaging device to perform the target scan on the target subject within the scanning angle range. The method may include generating a plurality of reconstruction images corresponding to the plurality of reconstruction angle ranges based on the scan data.

In some embodiments, at least one overlapping angle range may exist between at least one pair of adjacent reconstruction angle ranges of the plurality of reconstruction angle ranges, one overlapping angle range corresponding to one of the at least one pair of adjacent reconstruction angle ranges.

In some embodiments, the at least one overlapping angel range may include a first overlapping angel range between a first pair of adjacent reconstruction angle ranges and a second overlapping angel range between a second pair of adjacent reconstruction angle ranges. A span of the first overlapping angel range may be different from a span of the second overlapping angel range.

In some embodiments, the obtaining scan data may include obtaining real-time scan data collected by the imaging device during the target scan. The generating a plurality of reconstruction images corresponding to the plurality of reconstruction angle ranges based on the scan data may include for each of the plurality of reconstruction images, starting image reconstruction based on the real-time scan data after the imaging device rotates to a starting angle of the corresponding reconstruction angle range.

In some embodiments, the target scan may be a perfusion scan. The determining a target temporal resolution may include obtaining reference information relating to a contrast agent used in the perfusion scan and determining the target temporal resolution based on the reference information. The reference information may include at least one of a contrast agent concentration of the contrast agent, an injection volume, an injection speed, a development stage of the contrast agent, or accuracy requirement of perfusion parameters.

In some embodiments, the determining a plurality of reconstruction angle ranges may include obtaining a rotation speed of the imaging device during the target scan, determining a reference value of a step based on the rotation speed and the target temporal resolution, the step being an angle interval between adjacent reconstruction angle ranges among the plurality of reconstruction angle ranges, and determining the plurality of reconstruction angle ranges based on the reference value.

In some embodiments, the reference value of the step may include a plurality of reference values, and the angle interval between adjacent reconstruction angle ranges among the plurality of reconstruction angle ranges may be changing.

In some embodiments, the target scan may be a perfusion scan. In some embodiments, the target temporal resolution may include a plurality of target temporal resolutions corresponding to a plurality of development stages of a contrast agent used in the perfusion scan, and each of the plurality of reference values may be determined based on one of the plurality of target temporal resolutions and the rotation speed.

In some embodiments, the method may further include generating one or more predicted reconstruction images based on the plurality of reconstruction images. Each of the one or more predicted reconstruction images may correspond to a reconstruction angle range that is between two adjacent reconstruction angle ranges among the plurality of reconstruction angle ranges.

In some embodiments, the generating the one or more predicted reconstruction images based on the plurality of reconstruction images may include obtaining a first trained image prediction model and generating the one or more predicted reconstruction images by inputting the plurality of reconstruction images into the first trained image prediction model.

In some embodiments, the generating the one or more predicted reconstruction images based on the plurality of reconstruction images may include obtaining at least one pair of adjacent reconstruction images among the plurality of reconstruction images, obtaining a second trained image prediction model, and for each of the at least one pair of adjacent reconstruction images, generating a predicted reconstruction image by inputting the pair of adjacent reconstruction images into the second trained image prediction model.

In some embodiments, a temporal resolution of the plurality of reconstruction images may be equal to or higher than the target temporal resolution.

In another aspect of the present disclosure, a non-transitory computer readable medium is provided. The non-transitory computer readable medium may include executable instructions that, when executed by at least one processor, direct the at least one processor to perform a method for medical image reconstruction. The method may include determining a target temporal resolution of a target scan on a target subject within a scanning angle range. The method may include determining, based on the target temporal resolution, a plurality of reconstruction angle ranges each of which being within the scanning angle range. The method may include obtaining scan data acquired by causing an imaging device to perform the target scan on the target subject within the scanning angle range. The method may include generating a plurality of reconstruction images corresponding to the plurality of reconstruction angle ranges based on the scan data . . .

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2A and FIG. 2B are block diagrams illustrating exemplary processing devices according to some embodiments of the present disclosure;

FIG. 3A is a flowchart illustrating an exemplary process for image reconstruction according to some embodiments of the present disclosure;

FIG. 3B illustrates an exemplary reconstruction angle range according to some embodiments of the present disclosure;

FIG. 3C illustrates a plurality of exemplary reconstruction angle ranges according to some embodiments of the present disclosure;

FIG. 4 is a flowchart illustrating an exemplary process for determining a plurality of reconstruction angle ranges according to some embodiments of the present disclosure;

FIG. 5 is a flowchart illustrating an exemplary process for generating one or more predicted reconstruction images according to some embodiments of the present disclosure; and

FIG. 6 is a flowchart illustrating an exemplary process for generating a predicted reconstruction image according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

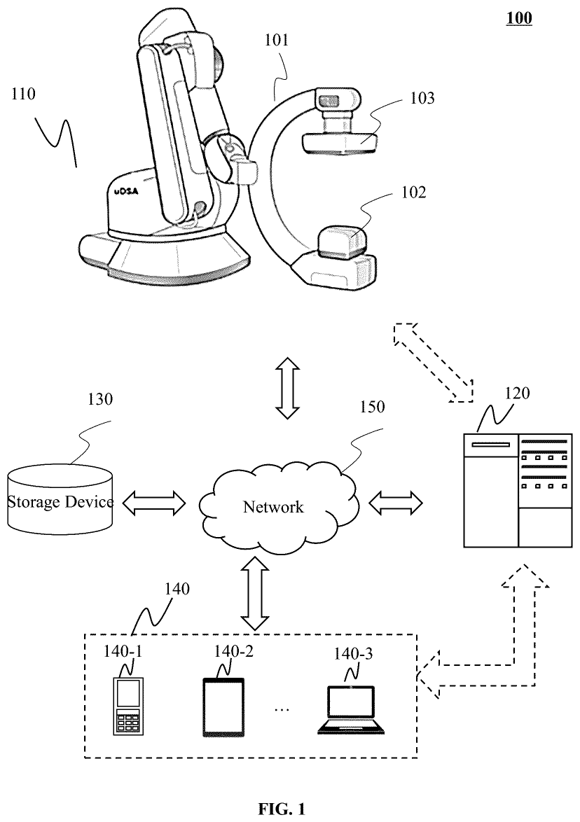

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices (e.g., processing device 120 as illustrated in FIG. 1) may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “image” in the present disclosure is used to collectively refer to image data (e.g., scan data) and/or images of various forms, including a two-dimensional (2D) image, a three-dimensional (3D) image, a four-dimensional (4D) image, etc. The term “pixel” and “voxel” in the present disclosure are used interchangeably to refer to an element of an image.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The term “imaging modality” or “modality” as used herein broadly refers to an imaging method or technology that gathers, generates, processes, and/or analyzes imaging information of a subject. The subject may include a biological subject (e.g., a human, an animal), a non-biological subject (e.g., a phantom), etc. In some embodiments, the subject may include a specific part, organ, and/or tissue of the subject. For example, the subject may include head, brain, neck, body, shoulder, arm, thorax, cardiac, stomach, blood vessel, soft tissue, knee, feet, or the like, or any combination thereof. The term “object” or “subject” are used interchangeably.

Perfusion scan generally requires relatively high temporal resolution, such as 2s. A CT device is often used to perform perfusion scans. Compared to a traditional CT scan, the scan speed of a CBCT (Cone beam Computer Tomography) scan is relatively slower. The scan speed of a CBCT scan is affected by the data collection rate of a flat panel detector and the rotation speed of a gantry, which limits the application of CBCT in perfusion imaging. The present disclosure proposes systems and methods for image reconstruction that can improve the temporal resolution of CBCT scans so that CBCT scans can be used in perfusion imaging.

An aspect of the present disclosure relates to systems and methods for medical image reconstruction. The systems and methods may determine a target temporal resolution of a target scan on a target subject within a scanning angle range. The systems and methods may determine, based on the target temporal resolution, a plurality of reconstruction angle ranges each of which being within the scanning angle range. The systems and methods may generate a plurality of reconstruction images corresponding to the plurality of reconstruction angle ranges by causing an imaging device to perform the target scan on the target subject within the scanning angle range, a temporal resolution of the plurality of reconstruction images being equal to or higher than the target temporal resolution.

According to some embodiments of the present disclosure, the plurality of reconstruction angle ranges may be selected from the scanning angle range according to the target temporal resolution, and the plurality of reconstruction images corresponding to the plurality of reconstruction angle ranges may be generated. That is, by performing one scan, the plurality of reconstruction images may be obtained, thereby improving the utilization of the scanning angle range and reducing the scanning time duration of the target subject. In some embodiments, the target scan may be a perfusion scan. Thus, the plurality of reconstruction images fulfilling the target temporal resolution may be generated by performing one scan and thus only need to inject a contrast agent into the target subject once, thereby reducing injection times to the target subject and the scanning time duration of the target subject.

FIG. 1 is a schematic diagram illustrating an exemplary imaging system 100 according to some embodiments of the present disclosure. In some embodiments, the imaging system 100 may be configured for non-invasive biomedical imaging, such as for disease diagnostic, treatment, and/or research purposes. In some embodiments, the imaging system 100 may be used for perfusion imaging.

In some embodiments, the imaging system may include a single modality imaging system and/or a multi-modality imaging system. The single modality imaging system may include, for example, an X-ray imaging system, a computed tomography (CT) system (e.g., a spiral CT system, a cone-beam CT system, etc.), a single photon emission computed tomography (SPECT) system, a digital radiography (DR) system, or the like, or any combination thereof. The multi-modality imaging system may include, for example, a positron emission tomography-CT (PET-CT) system, an X-ray imaging-magnetic resonance imaging (X-ray-MRI) system, a SPECT-MRI system, a CT guided radiotherapy system, etc. It should be noted that the imaging system described below is merely provided for illustration purposes, and not intended to limit the scope of the present disclosure.

In some embodiments, the imaging system 100 may include modules and/or components for performing imaging and/or related analysis. Merely by way of example, as illustrated in FIG. 1, the imaging system 100 may include a medical imaging device 110, a processing device 120, a storage device 130, one or more terminals 140, and a network 150. For brevity, the medical imaging device 110 is also referred to as imaging device 110. The components in the imaging system 100 may be connected in various ways. Merely by way of example, the imaging device 110 may be connected to the processing device 120 through the network 150 or directly as illustrated in FIG. 1. As another example, the terminal(s) 140 may be connected to the processing device 120 via the network 150 or directly as illustrated in FIG. 1.

The imaging device 110 may be configured to acquire imaging data relating to a target subject. The imaging data relating to a target subject may include an image (e.g., an image slice), projection data, or a combination thereof. In some embodiments, the imaging data may be two-dimensional (2D) imaging data, three-dimensional (3D) imaging data, four-dimensional (4D) imaging data, or the like, or any combination thereof. In some embodiments, the imaging device 110 may include a CT device (e.g., a CT device), an X-ray imaging device, a DR device, a SPECT device, a PET-CT device, an X-ray-MRI device, a SPECT-MRI device, a CT guided radiotherapy device.

The following descriptions are provided with reference to a cone-beam CT (CBCT) device for example. It is understood that this is for illustration purposes and not intended to be limiting. Merely as an example, a CBCT device with a C-shape support is shown in FIG. 1. In some embodiments, the CBCT device can perform perfusion imaging. As shown in FIG. 1, the CBCT device may include a scanning support 101, a radiation source 102, and a detector 103. The radiation source 102 may include a tube configured to emit radioactive rays (e.g., X-rays) toward the target subject. The detector 103 may detect radioactive rays (e.g., X-rays) after passing through the target subject. In some embodiments, the detector 103 may be a flat-panel detector or a non-plate detector. The radiation source 102 and the detector 103 may be mounted on the scanning support 101. The scanning support 101 is used to drive the radiation source 102 and the detector 103 to rotate around the target subject. In some embodiments, the rotation mode of the scanning support 101 may include longitudinal rotation. For example, when the target subject is lying on a scanning table, the scanning support 101 may drive the radiation source 102 and the detector 103 to rotate around the scanning table. In some embodiments, the rotation mode of the scanning support 101 may include horizontal rotation. The rotation mode of the scanning support 101 is not limited. The detector 103 may be used to obtain the projection data of the scanned target subject. The obtained projection data may be used to reconstruction an image of the target subject. The image may be a two-dimensional image, a three-dimensional image, or a four-dimensional image.

The processing device 120 may process data and/or information obtained from the imaging device 110, the terminal(s) 140, and/or the storage device 130. For example, the processing device 120 may determine a target temporal resolution of a target scan on a target subject within a scanning angle range. As another example, the processing device 120 may determine, based on the target temporal resolution, a plurality of reconstruction angle ranges each of which being within the scanning angle range. As still an example, the processing device 120 may generate a plurality of reconstruction images corresponding to the plurality of reconstruction angle ranges based on the scan data. A temporal resolution of the plurality of reconstruction images may be equal to or higher than the target temporal resolution.

In some embodiments, the processing device 120 may be a computer, a user console, a single server or a server group, etc. The server group may be centralized or distributed. In some embodiments, the processing device 120 may be local or remote. For example, the processing device 120 may access information and/or data stored in the imaging device 110, the terminal(s) 140, and/or the storage device 130 via the network 150. As another example, the processing device 120 may be directly connected to the imaging device 110, the terminal(s) 140 and/or the storage device 130 to access stored information and/or data. In some embodiments, the processing device 120 may be implemented on a cloud platform. Merely by way of example, the cloud platform may include a private cloud, a public cloud, a hybrid cloud, a community cloud, a distributed cloud, an inter-cloud, a multi-cloud, or the like, or any combination thereof.

The storage device 130 may store data, instructions, and/or any other information. In some embodiments, the storage device 130 may store data obtained from the terminal(s) 140 and/or the processing device 120. For example, the storage device 130 may store a rotation speed of the imaging device. As another example, the storage device 130 may store a first trained image prediction model and/or a second trained image prediction model. In some embodiments, the storage device 130 may store data and/or instructions that the processing device 120 may execute or use to perform exemplary methods/systems described in the present disclosure. In some embodiments, the storage device 130 may include a mass storage device, a removable storage device, a volatile read-and-write memory, a read-only memory (ROM), a cloud storage device, or the like, or any combination thereof. In some embodiments, the storage device 130 may be part of the processing device 120.

In some embodiments, a user (e.g., a doctor, a technician, or an operator) may interact with the imaging system 100 through the terminal(s) 140. For example, the user may input a target temporal resolution and/or view reconstruction images via the terminal 140. In some embodiments, the terminal(s) 140 may include a mobile device 140-1, a tablet computer 140-2, a laptop computer 140-3, or the like, or any combination thereof. In some embodiments, the terminal(s) 140 may be part of the processing device 120.

The network 150 may include any suitable network that can facilitate the exchange of information and/or data for the imaging system 100. In some embodiments, one or more components of the imaging device 110 (e.g., a CT device, a PET device, etc.), the terminal(s) 140, the processing device 120, the storage device 130, etc., may communicate information and/or data with one or more other components of the imaging system 100 via the network 150. As another example, the processing device 120 may obtain user instructions from the terminal(s) 140 via the network 150. The network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (“VPN”), a satellite network, a telephone network, routers, hubs, switches, server computers, and/or any combination thereof.

It should be noted that the above description of the imaging system 100 is merely provided for the purposes of illustration, and not intended to limit the scope of the present disclosure. For persons having ordinary skills in the art, multiple variations and modifications may be made under the teachings of the present disclosure. For example, the imaging system 100 may include one or more additional components and/or one or more components of the imaging system 100 described above may be omitted. Additionally or alternatively, two or more components of the imaging system 100 may be integrated into a single component. A component of the imaging system 100 may be implemented on two or more sub-components.

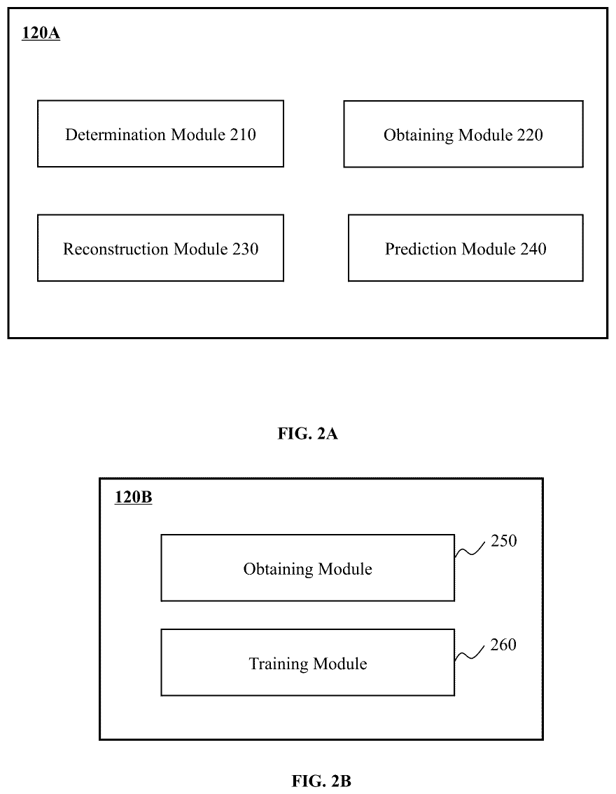

FIG. 2A and FIG. 2B are block diagrams illustrating exemplary processing devices 120A and 120B according to some embodiments of the present disclosure. In some embodiments, the processing devices 120A and 120B may be embodiments of the processing device 120 as described in connection with FIG. 1. In some embodiments, the processing devices 120A and 120B may be respectively implemented on a processor. For example, the processing device 120B may be implemented on a first processor of a vendor who provides and/or maintains one or more machine learning models, while the processing device 120A may be implemented on a second processor of a user of the machine learning model(s). Alternatively, the processing devices 120A and 120B may be implemented on a same processor.

As shown in FIG. 2A, the processing device 120A may include a determination module 210, an obtaining module 220, a reconstruction module 230, and a prediction module 240.

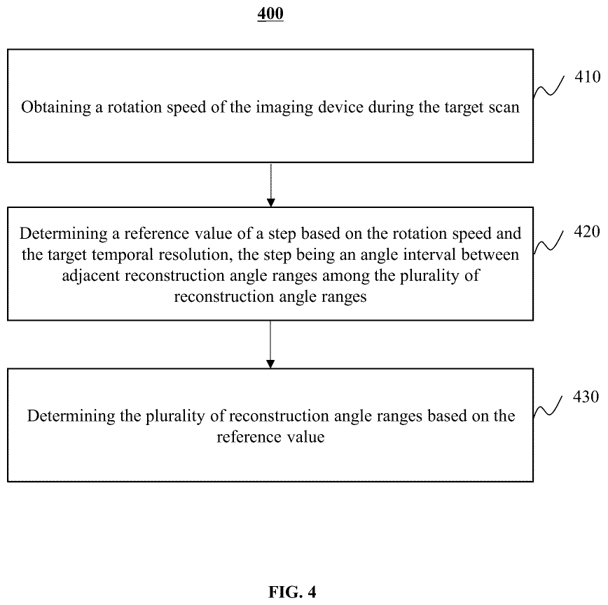

The determination module 210 may be configured to determine a target temporal resolution of a target scan on a target subject within a scanning angle range. The determination module 210 may be further configured to determine, based on the target temporal resolution, a plurality of reconstruction angle ranges each of which being within the scanning angle range. More descriptions regarding the target temporal resolution and the plurality of reconstruction angle ranges may be found elsewhere in the present disclosure (e.g., operations 310 and 320, and FIG. 5, and the description thereof).

The obtaining module 220 may be configured to obtain scan data acquired by causing an imaging device to perform the target scan on the target subject within the scanning angle range. More descriptions regarding obtaining the scan data may be found elsewhere in the present disclosure (e.g., operations 330 and the description thereof).

The reconstruction module 230 may be configured to generate a plurality of reconstruction images corresponding to the plurality of reconstruction angle ranges based on the scan data. More descriptions regarding the plurality of reconstruction images may be found elsewhere in the present disclosure (e.g., operation 340 and the description thereof).

The prediction module 240 may be configured to generate one or more predicted reconstruction images based on the plurality of reconstruction images. Each of the one or more predicted reconstruction images may correspond to a reconstruction angle range that is between two adjacent reconstruction angle ranges among the plurality of reconstruction angle ranges. More descriptions regarding the predicted reconstruction images may be found elsewhere in the present disclosure (e.g., operation 350 and the descriptions thereof).

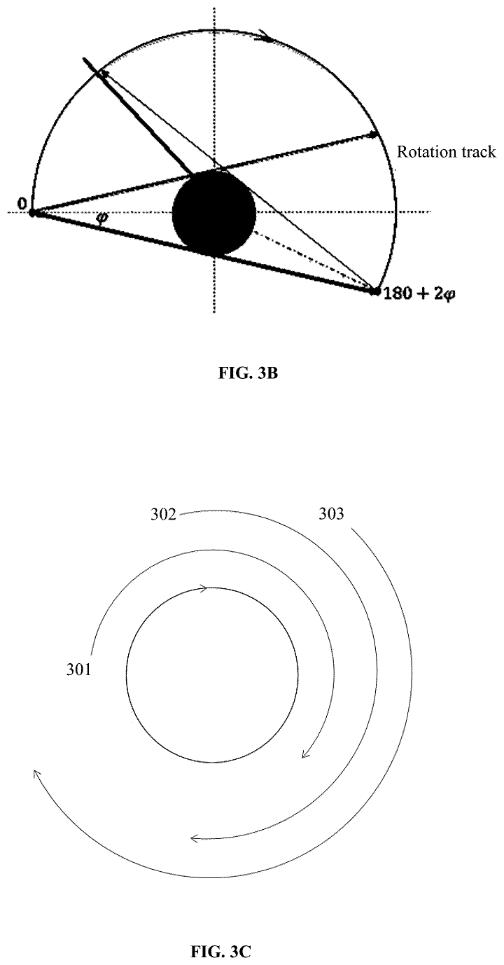

As shown in FIG. 4B, the processing device 120B may include an obtaining module 250 and a training module 260.

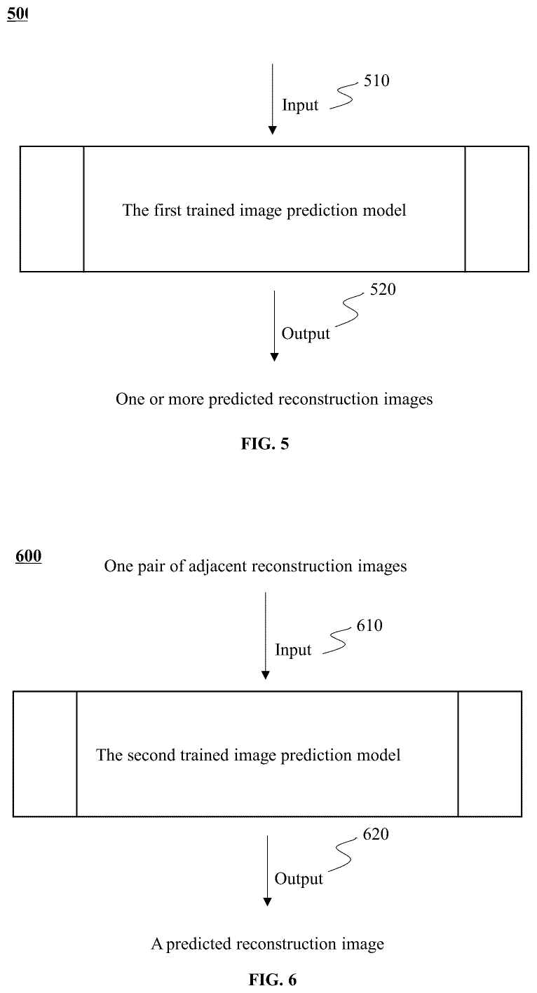

The obtaining module 250 may be configured to obtain data/information used for model training. For example, the obtaining module 250 may obtain a plurality of first training samples and a first preliminary model used for generating a first trained image prediction model. As another example, the obtaining model 240 may obtain a plurality of second training samples and a second preliminary model used for generating a second trained image prediction model. More descriptions regarding the training samples and/or the and the preliminary models may be found elsewhere in the present disclosure (e.g., FIGS. 5 and 6 and the descriptions thereof).

The training module 260 may be configured to generate a trained model (e.g., the first trained image prediction model and/or the second trained image prediction model). For example, the training module 260 may generate the first trained image prediction model by training the first preliminary model using the plurality of first training samples. As another example, the training module 260 may generate the second trained image prediction model by training the second preliminary model using the plurality of second training samples. More descriptions regarding the training process(es) may be found elsewhere in the present disclosure (e.g., FIGS. 5 and 6 and the descriptions thereof).

It should be noted that the above description is merely provided for the purposes of illustration, and not intended to limit the scope of the present disclosure. Apparently, for persons having ordinary skills in the art, multiple variations and modifications may be conducted under the teachings of the present disclosure. However, those variations and modifications do not depart from the scope of the present disclosure. Each of the modules described above may be a hardware circuit that is designed to perform certain actions, e.g., according to a set of instructions stored in one or more storage media, and/or any combination of the hardware circuit and the one or more storage media.

In some embodiments, the processing device 120A and/or the processing device 120B may share two or more of the modules, and any one of the modules may be divided into two or more units. For instance, the processing device 120A and the processing device 120B may share one obtaining module. In some embodiments, the processing device 120A and/or the processing device 120B may include one or more additional modules, such as a storage module (not shown) for storing data. In some embodiments, the processing device 120A and the processing device 120B may be integrated into one processing device 120. In some embodiments, the generation of the first trained image prediction model and the generation of the second trained image prediction model may be achieved by two different processing devices 120B respectively. In some embodiments, a module in the processing device 120A and/or the processing device 120B may be omitted. For instance, the prediction module 240 may be omitted.

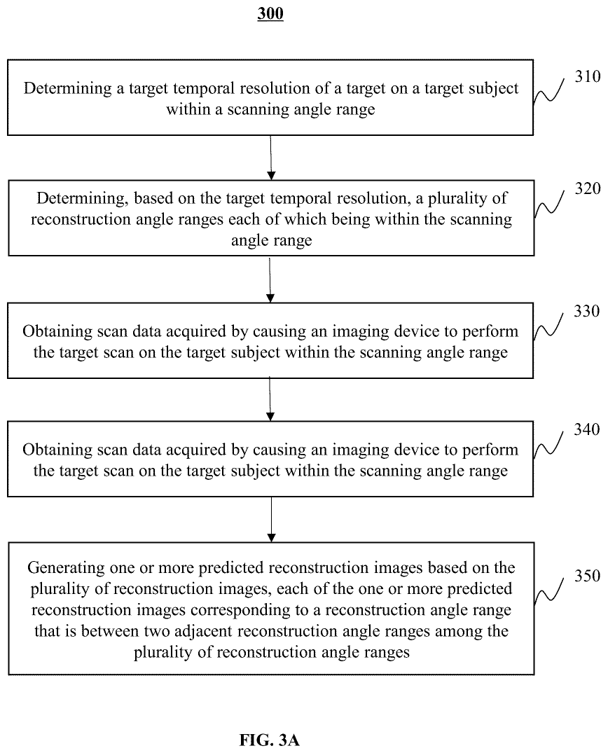

FIG. 3A is a flowchart illustrating an exemplary process for image reconstruction according to some embodiments of the present disclosure. In some embodiments, process 300 may be implemented as a set of instructions (e.g., an application) stored in a storage device (e.g., the storage device 130). The processing device 120A (e.g., one or more modules illustrated in FIG. 2A) may execute the set of instructions, and when executing the instructions, the processing device 120A may be configured to perform the process 300.

In 310, the processing device 120A (e.g., the determination module 210) may determine a target temporal resolution of a target scan on a target subject (e.g., a patient) within a scanning angle range.

The temporal resolution refers to the amount of time needed to obtain a subset of scan data used to reconstruct a reconstruction image of the target subject. For example, a time period may start from a time point when the collection of a previous subset of scan data is completed and end at a time point when the collection of a current subset of scan data is completed, and the temporal resolution is the duration of this time period. As another example, a time period may start from a time point when the collection of a subset of scan data starts and end at a time point when the collection of the subset of scan data is completed, and the temporal resolution is the duration of this time period. In some embodiments, the reconstruction image may be constructed or generated in real-time. The temporal resolution may refer to the amount of time needed to generate a reconstruction image of the target subject. For example, a time period may start from a time point when the reconstruction of a previous reconstruction image is completed and end at a time point when the reconstruction of a current reconstruction image is completed, and the temporal resolution is the duration of this time period. As another example, a time period may start from a time point when the reconstruction of a reconstruction image starts and end at a time point when the reconstruction of the reconstruction image is completed, and the temporal resolution is the duration of this time period.

The target temporal resolution may refer to a minimum value or a threshold of the temporal resolution. In some embodiments, the target temporal resolution may be constant during the target scan, that is, there is one target temporal resolution, such as 1s, 1.5s, 2s, 3s, 4s, 5s, and so on. Merely as an example, if the target temporal resolution is 2s, a reconstruction image may be generated regularly every 2s or less than 2s (e.g., 1s) during the target scan; the target temporal resolution is 3s, a reconstruction image may be generated regularly every 3s or less than 3s (e.g., 2s) during the target scan. The higher the target temporal resolution, the more frequently the reconstruction image may be generated.

In some embodiments, the target temporal resolution may change during the target scan, that is, there are multiple target temporal resolutions. For example, in a first stage of the target scan, the target temporal resolution may be 3s and a reconstruction image may be generated regularly every 3s; in a second stage of the target scan, the target temporal resolution may be 2s and a reconstruction image may be generated regularly every 2s.

In some embodiments, the processing device 120A may determine the target temporal resolution based on an input received from the terminal device 140. For example, a technical may input the target temporal resolution via the terminal device 140. In some embodiments, the processing device 120A may determine the target temporal resolution based on a system default setting. In some embodiments, the processing device 120A may determine the target temporal resolution based on information relating to the target subject and/or the target scan. The information may include at least one of a region of interest of the target subject, a type of the contrast agent, a contrast agent concentration of the contrast agent, an injection volume, an injection speed, a development stage of the contrast agent, accuracy requirement of perfusion parameters, scanning parameters, and so on. For example, the processing device 120A may obtain the information, and determine a target temporal resolution based on a corresponding relationship between the target temporal resolution and the information.

The target subject may include a biological subject (e.g., a human, an animal), a non-biological subject (e.g., a phantom), etc. In some embodiments, the target subject may include a specific part, organ, and/or tissue of the subject. For example, the target subject may include head, brain, neck, body, shoulder, arm, thorax, cardiac, stomach, blood vessel, soft tissue, knee, feet, or the like, or any combination thereof.

In some embodiments, the target scan may be a CBCT scan. In some embodiments, the target scan may be performed by the CBCT device when the target subject is not injected with a contrast agent. In some embodiments, the target scan may be a perfusion scan performed by the CBCT device when the target subject is injected with a contrast agent.

The scanning angle range may refer to an angle range for scanning the target subject using an imaging device. In some embodiments, the target scan may be a static scan, for example, a static perfusion scanning. In the static scan, a scanning bed or the target subject does not move during the scanning process while the detector and the radiation source rotate around the target subject. The scanning angle range may refer to the rotation angle range of the detector and the radiation source.

In some embodiments, before the target scan, the scanning angle range may be determined. In some embodiments, the scanning angle range may be determined according to the structural characteristics of the target subject or a region of interest of the target subject. In some embodiments, the scanning angle range may include a limited angle range, such as 0-360°, 0-400°, 0-440°, 0-480°, 0-540°, 0-1080°, and so on. In some embodiments, the scanning angle range may be determined during the target scan, for example, the target scan may be completed after a certain count of subsets of scan data used to reconstruct reconstruction images of the target subject are obtained.

In some embodiments, the target scan may be a perfusion scan. The processing device 120A may obtain reference information relating to a contrast agent used in the perfusion scan. The reference information may include at least one of a contrast agent concentration, an injection volume, an injection speed, a development stage of the contrast agent, accuracy requirement of perfusion parameters, or the like, or any combination thereof. The processing device 120A may determine the target temporal resolution based on the reference information.

Generally, the injection of the contrast agent into the target subject may be performed according to certain injection parameters, such as the contrast agent concentration, injection volume, and injection speed. Different injection parameters may lead to different requirements on the target temporal resolution.

Specifically, the contrast agent concentration of the contrast agent may affect the imaging effect of the contrast agent in the tissue or the lesion. For example, the contrast agent with higher concentration may have a more obvious imaging effect in the tissue or the lesion, so a higher target temporal resolution can be set to obtain high-quality reconstruction results (i.e., reconstruction images) can be obtained in a relatively shorter time.

Similarly, the injection volume may also affect the setting of the target temporal resolution. Specifically, when the injection volume is small, it may be necessary to obtain the reconstruction results in a short time, so it is necessary to set a higher target temporal resolution.

The injection speed of the contrast agent may be, for example, 1.5 mL/s, 2 mL/s, and so on. Under different injection speeds, the flow velocities of the contrast agent in tissues or the lesion are different, and different target temporal resolutions may be needed. For example, the faster the injection speed is, the higher the target temporal resolution is required, so that the reconstruction results can be obtained in a shorter time. Therefore, the target temporal resolution determined according to the injection parameters is more in line with the actual needs of a user.

In some embodiments, the target temporal resolution may be determined based on the development stage of the contrast agent. The contrast agent often needs to go through several development stages when it is injected into the target subject. In different development stages, the flow speeds and imaging effects of the contrast agent are often different. In some embodiments, the development stage of the contrast agent may include a flat scan phase, a slow inflow phase, a high-dose arterial phase, a low-dose outflow phase, a slow outflow phase, and other phases. The flat scan phase and the slow outflow phase are the beginning and end stages of the contrast agent entering the tissue or the lesion, and the flow rate in these two phases is often slow. Therefore, a low target temporal resolution (e.g., 4-5s) may be set for the flat scan phase and the slow outflow phase. The high-dose arterial phase is the phase with the fastest contrast agent flow velocity. Therefore, a high target temporal resolution (e.g., 1.5-2s) may be set for the high-dose arterial phase. The contrast agent flow velocity in the slow inflow phase and the low-dose outflow phase is moderate. Therefore, a moderate target temporal resolution (2-3s) may be set for the slow inflow phase and the low-dose outflow phase. In other words, different target temporal resolutions may be set to meet different requirements in multiple development stages of the contrast agent. Based on the different flow velocities and imaging effects of the contrast agent in each development phase, different target temporal resolutions may be set for different phases. In this way, the target temporal resolution may be flexibly adjusted based on the development stage of the contrast agent to improve the quality of the reconstruction results and avoid bringing too much scanning radiation to the target subject.

In some embodiments, the target temporal resolution may be determined according to the accuracy requirement of perfusion parameters. The accuracy requirement of perfusion parameters refers to the requirement for the accuracy of the perfusion parameters to be determined. The perfusion scan is often performed to obtain a time-density curve (TDC) of each voxel of the tissue or organ of the target subject by continuously scanning the target subject. The TDC may reflect the inflow and outflow process of the contrast agent in the tissue or organ (i.e., the blood perfusion process), and various perfusion parameters may be determined based on the TDC using different data models, thus generating a perfusion parameter map. In some embodiments, the perfusion scan may be a brain perfusion scan. The perfusion parameters may include a cerebral blood volume (CBV), a cerebral blood flow (CBF), a mean transit time (MTT), a time to peak (TTP) of the contrast agent, etc. The higher the target temporal resolution, the higher the accuracy of the generated perfusion parameters. In some embodiments, a higher target temporal resolution may be set if the requirement for the accuracy of the perfusion parameters is high. In some embodiments, the accuracy requirements may be represented by an accuracy threshold, such as 96%, 98%, etc.

In some embodiments, the target temporal resolution may be related to the heart function of the target subject. The processing device 120A may determine the target temporal resolution further based on the heart function of the target subject. Specifically, when the heart function of the target subject is poor, it may be necessary to set a higher target temporal resolution to obtain detailed perfusion parameters of the target subject.

In some embodiments, the processing device 120A may determine the target temporal resolution based on the reference information and a reference value of the target temporal resolution. The reference value may correspond to a reference scan that is performed based on preset injection parameters and preset accuracy requirement of perfusion parameters. In some embodiments, the processing device 120A may adjust the reference value of the target temporal resolution based on a plurality of differences between the target scan and the reference scan. T differences may include a difference between an injection parameter of the target scan and a corresponding preset injection parameter of the reference scan, a difference between the accuracy requirement of the target scan and the preset accuracy requirement of the reference scan, or the like. In some embodiments, each of the plurality of differences may be assigned with a weight coefficient. The weight coefficients of different differences may be different. The processing device 120A may adjust the reference value of the target temporal resolution based on the plurality of differences and their weight coefficients.

In some embodiments, the processing device 120A may determine the target temporal resolution based on the reference information using a trained prediction model. The trained prediction model may be a machine learning model with the reference information as a model input and the target temporal resolution as a model output.

In some embodiments, after the processing device 120A determine the target temporal resolution, the technical or the engineer may adjust the target temporal resolution, and the adjusted target temporal resolution may be designated as the final target temporal resolution.

By determining the target temporal resolution based on the reference information, the characteristics of the contrast agent, the accuracy requirement, the condition of the target subject can be considered, thereby improving the accuracy of the determined target temporal resolution and the scanning efficiency.

In 320, the processing device 120A (e.g., the determination module 210) may determine, based on the target temporal resolution, a plurality of reconstruction angle ranges each of which being within the scanning angle range.

A reconstruction angle range may be part of the scanning angle range, and scan data collected in the reconstruction angle range may be used to generate one reconstruction image. In some embodiments, the span of each reconstruction angle range may be greater than a span threshold (or referred to as a minimum reconstruction angle range). The span threshold may be determined according to the reconstruction algorithm to be used to reconstruct the reconstruction images. In some embodiments, the span threshold may be equal to 180 degrees or greater than 180 degrees.

For illustration purposes, the following descriptions take the filtered backprojection reconstruction method as an example to describe the determination of the span threshold. In the filtered backprojection reconstruction method, Randon transform is performed on sinogram, and the period of the sinogram is π, so it is necessary to obtain the 180° line integral of each point in the reconstruction field of view (FOV) to complete the reconstruction. As shown in FIG. 3B, in CBCT scanning, the 180° line integral projection data of the scanned object can be obtained only when the radiation source rotates at least (180°+2φ) around the reconstruction FOV corresponding to the scanned object (the black circular area in the middle). That is, the span threshold of the filtered backprojection reconstruction method may be 180°+2φ. As shown in FIG. 3B, the sector angle 20 may be set according to the radius R of the reconstruction FOV of the scanned object and the distance L between the radiation source and the center of reconstruction FOV, such as φ=arc sin (R/L). In some embodiments, the reconstruction angle range may be determined based on other reconstruction methods.

In the present application, the plurality of reconstruction angle ranges may be selected from the scanning angle range to obtain a plurality of reconstruction results (e.g., reconstruction images) based on scan data collected in one scan, which improves the utilization of the scan data. In some embodiments, the plurality of reconstruction angle ranges may be selected in the scanning angle range according to a reference value of a step. The step may be an angle interval between adjacent reconstruction angle ranges among the plurality of reconstruction angle ranges. For example, as shown in FIG. 3C, the scanning angle range may be 0-360°, the span of each reconstruction angle range may be (π+20°=200°), and the step may be 80°, then the plurality of reconstruction angle ranges may include a reconstruction angle range 301 (0-200°), a reconstruction angle range 302 (80°-280°), and a reconstruction angle range 303 (160°-360°). In some embodiments, it is also possible to arbitrarily select a plurality of reconstruction angle ranges from the scanning angle range under the premise of meeting the target temporal resolution.

In some embodiments, at least one overlapping angle range may exist between at least one pair of adjacent reconstruction angle ranges of the plurality of reconstruction angle ranges, one overlapping angle range corresponding to one of the at least one pair of adjacent reconstruction angle ranges. For example, there may be N reconstruction angle ranges. N may be an integer greater than 1, for example, 2, 3, 4, 5, 6, and so on. Thus, there may be (N-1) pairs of adjacent reconstruction angle ranges. The two scanning angle ranges in each pair of adjacent reconstruction angle ranges may be overlapped with each other or not. For the N-1 pairs of adjacent reconstruction angle ranges, there may be at least one overlapping angle ranges and at most N-1 overlapping angle ranges. Merely by way of example, the reconstruction angle ranges may include) (0-200°), (80°-280°), and (280°-480°). An overlapping angle range (i.e., (80°-200°)) exists between the reconstruction angle ranges (0-200°) and (80°-280°), while no overlapping angle range exists between the reconstruction angle ranges (80°-280°) and (280°-480°).

In some embodiments, one overlapping angle range may exist between each pair of adjacent reconstruction angle ranges of the plurality of reconstruction angle ranges. In some embodiments, the span of all overlapping angle ranges of the plurality of reconstruction angle ranges may be the same.

In some embodiments, the at least one overlapping angel range may include a first overlapping angel range between a first pair of adjacent reconstruction angle ranges and a second overlapping angel range between a second pair of adjacent reconstruction angle ranges. The span of the first overlapping angel range may be different from the span of the second overlapping angel range. In other words, the step of the reconstruction angle ranges may change. Merely by way of example, the reconstruction angle ranges may include a reconstruction angle range R1 (0-200°), a reconstruction angle range R2 (80°-280°), and a reconstruction angle range R3 (180°-380°). The reconstruction angle ranges R1 and R2 may form a first pair of adjacent reconstruction angle ranges with a first overlapping angel range (80°-200°). The reconstruction angle ranges R2 and R3 may form a second pair of adjacent reconstruction angle ranges with a second overlapping angel range (180°-280°). The span of the first overlapping angel range (80°-200° is 120°), while the span of the second overlapping angel range (180°-280° is 100°).

In some embodiments, the first overlapping angel range (or the first pair of adjacent reconstruction angle ranges) and the second overlapping angel range (or the second pair of adjacent reconstruction angle ranges) may correspond to different development stages of the contrast agent. For example, the first overlapping angel range may be larger than the second overlapping angel range. The first pair of adjacent reconstruction angle ranges may be used to generate reconstruction images corresponding to the flat scan phase or the slow outflow phase, and the second pair of adjacent reconstruction angle ranges may be used to generate reconstruction images corresponding to the slow inflow phase, the high-dose arterial phase, or the low-dose outflow phase. As another example, the first pair of adjacent reconstruction angle ranges may be used to generate reconstruction images corresponding to the slow inflow phase or the low-dose outflow phase, and the second pair of adjacent reconstruction angle ranges may be used to generate reconstruction images corresponding to the high-dose arterial phase.

In some embodiments, the first overlapping angel range may exist between each pair of a plurality of first pairs of adjacent reconstruction angle ranges. In some embodiments, the second overlapping angel range may exist between each pair of a plurality of second pairs of adjacent reconstruction angle ranges. In some embodiments, one or more other overlapping angel ranges different from the first overlapping angel range and the second overlapping angel range may exist between one or more other pairs of adjacent reconstruction angle ranges. In some embodiments, different development stages of the contrast agent may correspond to different overlapping angel ranges. For example, each of the flat scan phase, the slow inflow phase, the high-dose arterial phase, the low-dose outflow phase, the slow outflow phase, and other phases may correspond to different overlapping angel ranges, respectively. In some embodiments, the flat scan phase and the slow outflow phase may correspond to a same first overlapping angel range. The slow inflow phase and the low-dose outflow phase may correspond to a same second overlapping angel range. The high-dose arterial phase may correspond to a third overlapping angel range. The first overlapping angel range may be larger than the second overlapping angel range, and the second overlapping angel range may be larger than the third overlapping angel range.

In some embodiments, the development stages of the contrast agent may be determined or divided by a user. In some embodiments, the processing device 120A may determine the development stages of the contrast agent according to a plurality of reference images acquired using the contrast agent in other scans.

In some embodiments, the processing device 120A may determine the plurality of reconstruction angle ranges based on the target temporal resolution and a rotation speed of the imaging device during the target scan. More descriptions regarding determining the plurality of reconstruction angle ranges based on the target temporal resolution may be found elsewhere in the present disclosure, e.g., FIG. 4.

In 330, the processing device 120A (e.g., the obtaining module 220) may obtain scan data acquired by causing an imaging device to perform the target scan on the target subject within the scanning angle range.

In some embodiments, the processing device 120A may obtain the scan data from the imaging device. In some embodiments, the processing device 120A may send scan parameters of the target scan to the imaging device locally or remotely and cause the imaging device to perform the target scan on the target subject. The imaging device may collect scan data by performing the target scan on the target subject. The processing device 120A may obtain the scan data collected by the imaging device.

In some embodiments, the processing device 120A may obtain the scan data from the storage of another computing device or the cloud. The scan data may be acquired by causing the imaging device to perform the target scan on the target subject within the scanning angle range. For example, the imaging device may obtain scan parameters of the target scan from a computing device or the cloud and collect scan data by performing the target scan on the target subject. The imaging device may send the scan data to the computing device or upload the scan data to a cloud.

In some embodiments, the scan data may be the existing scan data. The existing scan data may be collected from a performed target scan, the temporal resolution of which meets the target temporal resolution. The processing device 120A may obtain the performed target scan.

In some embodiments, the processing device 120A may obtain real-time scan data collected by the imaging device during the target scan.

In 340, the processing device 120A (e.g., the reconstruction module 230) may generate a plurality of reconstruction images corresponding to the plurality of reconstruction angle ranges based on the scan data.

In some embodiments, after determining the plurality of reconstruction angle ranges, the processing device 120A may cause the imaging device to perform the target scan on the target subject within the scanning angle range. For example, the processing device 120A may send scan parameters to the imaging device and cause the imaging device to perform the target scan on the target subject. The imaging device may collect scan data by performing the target scan on the target subject.

In some embodiments, the scan data may include pieces of scan data collected at a plurality of time points, and each piece of scan data may be labeled by a time label or an angle label. The time label of a piece of scan data may indicate the point at which the piece of scan data is collected, and an angle label of the piece of scan data may indicate an angle of the radiation source with respect to a reference direction when the piece of scan data is collected. For each of the plurality of reconstruction angle ranges, the processing device 120A may obtain, from the scan data, a subset of scan data corresponding to the reconstruction angle range based on the angle label or the time label and the scan time corresponding to the reconstruction angle range. The processing device 120A may generate a reconstruction image based on the subset of scan data for each of the plurality reconstruction angle ranges.

A temporal resolution of the plurality of reconstruction images may refer to the amount of actual time to obtain a subset of scan data used to generate or reconstruct each of the plurality of reconstruction images. In some embodiments, the plurality of reconstruction images may be constructed or generated in real time. The temporal resolution of the plurality of reconstruction images may refer to the amount of actual time to generate each of the plurality of reconstruction images. In some embodiments, the temporal resolution of the plurality of reconstruction images may be equal to or higher than the target temporal resolution. In some embodiments, the temporal resolution of the plurality of reconstruction images may be smaller slightly than the target temporal resolution. “Slightly” used herein may refer that a deviation between a temporal resolution of a reconstruction image and the target temporal resolution is smaller than or equal to a threshold, e.g., 2%, 5%, 10%, etc.

In some embodiments, the processing device 120A may obtain real-time scan data collected by the imaging device during the target scan, and perform real-time image reconstruction during the target scan. For example, for a reconstruction image, the processing device 120A may start image reconstruction based on the real-time scan data after the imaging device rotates to a starting angle of the corresponding reconstruction angle range. The processing device 120A may continue image reconstruction along with the rotation of the imaging device in the corresponding reconstruction angle range. After the imaging device rotates to an end angle of the corresponding reconstruction angle range, image reconstruction based on the scan data collected from the starting angle to the end angle may be completed, and the reconstruction image may be generated.

In some embodiments, for each of the plurality of reconstruction images, the processing device 120A may start image reconstruction after a subset scan data of the corresponding reconstruction angle range is collected. In some embodiments, the processing device 120A may start image reconstruction based on the scan data after all the scan data are collected (i.e., after the target scan is finished).

Merely as an example, it is assumed that the scanning angle range is 0-360°, and the reconstruction angle ranges are 0°-200°, 80°-280°, and 160°-360°. For the first reconstruction angle range 0°-200°, the scan data (e.g., projection data) collected within the angle range 0-200° may be reconstructed in real time from 0°, and a first reconstruction result (e.g., a first reconstruction image) may be obtained when the imaging device is rotated to 200°. For the second reconstruction angle range 80°-280°, the scan data (e.g., projection data) collected within the angle range 80°-280° may be reconstructed in real time when the imaging device is rotated to 80°, and a second reconstruction result (e.g., a second reconstruction image) may be obtained when the imaging device is rotated to 280°. For the third reconstruction angle range 160°-360°, the scan data (e.g., projection data) collected within the angle range 160°-360° may be reconstructed in real time when the imaging device is rotated to 160°, and a third reconstruction result (e.g., a third reconstruction image) may be obtained when the imaging device is rotated to 360º.

In some embodiments, image reconstruction may be performed according to one or more reconstruction algorithms, such as s two-dimensional filtered back projection (FBP) algorithm, a three-dimensional Feldkamp-Davis-Kress (FDK) algorithm, or other analytic reconstruction algorithms.

In some embodiments, it takes a certain time for the imaging device to complete the rotation of a reconstruction angle range, especially for some imaging devices with slow rotation speed. Therefore, a reconstruction image may be associated with a specific time point to indicate the acquisition time (or referred to as the reconstruction time) of the reconstruction image. For example, for each of the plurality of reconstruction angle ranges, the processing device 120A may determine a time when the imaging device rotates to a center angle of the reconstruction angle range, and designate the time as the reconstruction time of the reconstruction image corresponding to the reconstruction angle range.

Merely as an example, for the first reconstruction angle range 0°-200° mentioned above, a first time when the imaging device rotates to 100° may be recorded and designated as the reconstruction time corresponding to the first reconstruction result. For the second reconstruction angle range 80°-280°, a second time when the imaging device rotates to 180° may be recorded and designated as the reconstruction time corresponding to the second reconstruction result. For the third reconstruction angle range 160°-360°, a third time when the imaging device rotates to 260° may be recorded and designated as the reconstruction time corresponding to the third reconstruction result.

In some embodiments, the reconstruction result corresponding to a reconstruction angle range may be associated with other times, such as the time when the imaging device rotates to the minimum angle within the reconstruction angle range, the time when the imaging device rotates to the maximum angle within the reconstruction, as long as the reconstruction times of the plurality of reconstruction angle ranges are determined in the same manner.

In some embodiments, the plurality of reconstruction images corresponding to the plurality of reconstruction angle ranges may be displayed via, for example, a user terminal. In some embodiments, the reconstruction images may be displayed in real time. For example, for the reconstruction image obtained in the range of 0-200°, the reconstruction image may be displayed in real time when the imaging device rotates to 200° or immediately after the imaging device rotates to 200°.

In 350, the processing device 120A (e.g., the prediction module 240) may generate one or more predicted reconstruction images based on the plurality of reconstruction images, each of the one or more predicted reconstruction images corresponding to a reconstruction angle range that is between two adjacent reconstruction angle ranges among the plurality of reconstruction angle ranges.

In some embodiments, the predicted reconstruction image(s) and the reconstruction images may form the final reconstruction image set with a higher temporal resolution than the reconstruction images, so as to meet a higher imaging requirement. For example, the plurality of reconstruction images may fulfill the target temporal resolution, however, the temporal resolution of the plurality of reconstruction images may not be enough to obtain some parameters relating to the target subject or the lesion. In some embodiments, the processing device 120A may generate one or more predicted reconstruction images based on the plurality of reconstruction images using an interpolation fitting method. For example, the processing device 120A may generate interpolation values based on a pair reconstruction images corresponding to adjacent reconstruction angle ranges and generate a predicted reconstruction image based on the interpolation values. In some embodiments, the processing device 120A may generate one or more predicted reconstruction images by inputting the plurality of reconstruction images into a first trained image prediction model. In some embodiments, the processing device 120A may generate a predicted reconstruction image by inputting a pair of adjacent reconstruction images into the second trained image prediction model. More descriptions regarding generating the one or more predicted reconstruction images or the predicted reconstruction image may be found elsewhere in the present disclosure, e.g., FIGS. 5 and 6.

In some embodiments, each of the one or more predicted reconstruction images may correspond to a reconstruction angle range that is between two adjacent reconstruction angle ranges among the plurality of reconstruction angle ranges. Merely as an example, a predicted reconstruction image may be generated based on two reconstruction images corresponding to reconstruction angle ranges 0-200° and 80°-280°, and the predicted reconstruction image may correspond to a reconstruction angle range of 40°-240°. As another example, two predicted reconstruction images may be generated based on three reconstruction images corresponding to reconstruction angle ranges 0-200°, 80°-280°, and 160°-360°, and the two predicted reconstruction images may correspond to reconstruction angle ranges of 40°-240° and 120°-320°.

In some embodiments, the processing device 120A may generate one or more interval reconstruction images based on the scan data. For example, the processing device 120A may determine one or more interval angle ranges between adjacent reconstruction angle ranges, and generate the interval reconstruction image(s) based on sets of scan data collected in the interval angle range(s). Merely as an example, the reconstruction angle ranges may include 0-200° and 80°-280°, and the processing device 120A may determine some interval angle range 10°-210°, 20°-220°, 30°-230°, 40°-240°, 50°-250°, 60°-260°, 70°-270°, and so on. The processing device 120A may then generate interval reconstruction images based on scan data collected in these interval angle ranges.

In some embodiments, the reconstruction images may be generated in real-time during the target scan, while the predicted reconstruction images and/or the interval reconstruction image(s) may be generated after the target scan is finished. For example, the reconstruction images may be generated and displayed to a scanning technician in real-time for real-time monitoring and/or adjustment of the target scan, and the predicted reconstruction images and/or the interval reconstruction image(s) may be generated after the target scan and displayed to a doctor with the reconstruction image for disease diagnosis. In this way, real-time image reconstruction with short latency can be achieved only using a small amount of computing resources, and more images can be provided to improve the accuracy of disease diagnosis.

It should be noted that the above description regarding the process 300 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, operation 350 may be omitted.

In some embodiments, the determination of the target temporal resolution and the reconstruction angle ranges may be performed when or after the target scan is performed within the scanning angle range. For example, the imaging device is directed to scan the target subject in the scanning angle range to collect scan data of the target subject. Then, the processing device 120A may determine the target temporal resolution and the reconstruction angle ranges, and generate the reconstruction images corresponding to the reconstruction angle ranges based on a plurality of subsets of the scan data collected in the reconstruction angle ranges.

FIG. 4 is a flowchart illustrating an exemplary process for determining a plurality of reconstruction angle ranges according to some embodiments of the present disclosure. In some embodiments, one or more operations of the process 400 may be performed to achieve at least part of operation 330 as described in connection with FIG. 3A.

In 410, the processing device 120A (e.g., the determination module 210) may obtain a rotation speed of the imaging device during the target scan.

The rotation speed of the imaging device refers to the rotation speed of the detector and the radiation source (or a gantry that supports the detector and the radiation source) during the target scan. The rotation speed of the imaging device may be predetermined (e.g., according to a system default setting) or set manually. In some embodiments, during the target scan, the rotation speed of the imaging device may be fixed. In some embodiments, during the target scan, the rotation speed of the imaging device may change with the rotation angle.

In 420, the processing device 120A (e.g., the determination module 210) may determine a reference value of a step based on the rotation speed and the target temporal resolution, the step being an angle interval between adjacent reconstruction angle ranges among the plurality of reconstruction angle ranges.

In some embodiments, the step may be an angle interval between two starting angels of the adjacent reconstruction angle ranges. In some embodiments, the step may be an angle interval between two ending angels of the adjacent reconstruction angle ranges. In some embodiments, the step may be an angle interval between two center angels of the adjacent reconstruction angle ranges.

In some embodiments, the processing device 120A may determine the reference value of the step based on the rotation speed and the target temporal resolution. For example, the processing device 120A may determine the reference value of the step by multiplying the rotation speed with the target temporal resolution. Merely as an example, the target temporal resolution may be 3s, and the rotation speed of the imaging device may be 50°/s, so it is necessary to obtain a reconstruction result at least every 150° (=50°/s×3s), that is, the reference value of the step may be 150°. After determining the reference value of the step, the step value of the plurality of reconstruction angle ranges may be determined, and the step value of the adjacent two reconstruction angle ranges may not be greater than the reference value of the step. For example, when the reference value of the step is 150°, the step value of the plurality of reconstruction angle range may be 150°, 120°, 100°, or other values less than 150°.

In some embodiments, the reference value of the step may include a plurality of reference values. The angle interval between adjacent reconstruction angle ranges among the plurality of reconstruction angle ranges may be changing. In some embodiments, the target temporal resolution may include a plurality of target temporal resolutions. For example, there may be multiple target temporal resolutions corresponding to a plurality of development stages of a contrast agent used in the perfusion scan. The processing device 120A may determine the plurality of reference values based on the plurality of target temporal resolutions and the rotation speed of the imaging device. Merely by way of example, the processing device 120A may determine the plurality of reference values by multiplying each of the plurality of target temporal resolutions with the rotation speed of the imaging device.

For illustration purposes, an example of determining reference values based on target temporal resolutions corresponding to a plurality of development stages of the contrast agent is provided. The plurality of development stages of the contrast agent may at least include a first development stage and a second development stage. The plurality of target temporal resolutions may at least include a first target temporal resolution and a second target temporal resolution. The processing device 120A may determine a first reference value based on the first target temporal resolution and the rotation speed, and determine a second reference value based on the second target temporal resolution and the rotation speed. When the contrast agent is in the first development stage, the step between adjacent reconstruction angle ranges may be equal to or smaller than the first reference value so that the temporal resolution of first reconstruction images collected in the first development stage may be equal to or greater than the first target temporal resolution; when the contrast agent is in the second development stage, the step between adjacent reconstruction angle ranges may be equal to or smaller than the second reference value so that the temporal resolution of second reconstruction images collected in the second development stage may be equal to or greater than the second target temporal resolution. For example, if the first target temporal resolution is greater than the second target temporal resolution, the first reference value may be smaller than the second reference value, and the temporal resolution of the first reconstruction images may be greater than that of the second reconstruction images. In this way, the temporal resolution of the reconstruction images can be dynamically adjusted to meet different imaging requirements of different development stages.

Merely as an example, the target temporal resolution may include five target temporal resolutions 1-5. The target temporal resolution 1 may correspond to the flat scan phase. The target temporal resolution 2 may correspond to the slow inflow phase. The target temporal resolution 3 may correspond to the high-dose arterial phase. The target temporal resolution 4 may correspond to the low-dose outflow phase. The target temporal resolution 5 may correspond to the slow outflow phase. The processing device 120A may determine a first reference value based on the target temporal resolution 1 and the rotation speed of the imaging device. The processing device 120A may determine a second reference value based on the target temporal resolution 2 and the rotation speed of the imaging device. The processing device 120A may determine a third reference value based on the target temporal resolution 3 and the rotation speed of the imaging device. The processing device 120A may determine a fourth reference value based on the target temporal resolution 4 and the rotation speed of the imaging device. The processing device 120A may determine a fifth reference value based on the target temporal resolution 5 and the rotation speed of the imaging device. In some embodiments, the target temporal resolution 1 and the target temporal resolution 5 may be the same. In some embodiments, the target temporal resolution 2 and the target temporal resolution 4 may be the same.

In 430, the processing device 120A (e.g., the determination module 210) may determine the plurality of reconstruction angle ranges based on the reference value.

In some embodiments, the processing device 120A may determine a first reconstruction angle range. The first reconstruction angle range may start from the rotation angle of 0°. The span of the first reconstruction angle range may be equal to or greater than the span threshold. Further, the processing device 120A may determine the step value of the plurality of reconstruction angle ranges, and the step value of the adjacent two reconstruction angle ranges may not be greater than the reference value of the step. The processing device 120A may determine a second reconstruction angle range by adding the step value to the first reconstruction angle range. Merely as an example, the first reconstruction angle range may be 0°-200° and the step value may be 80°. The second reconstruction angle range may be (0°+80°)-(200°+80°), i.e., 80°-280°. Similarly, the processing device 120A may determine other reconstruction angle ranges based on the reference value. For example, the processing device 120A may determine the third reconstruction angle range by adding the step value to the second reconstruction angle range.

In some embodiments, the reference value of the step may include a plurality of reference values. The processing device 120A may determine a reference value corresponding to a current reconstruction angle range from the plurality of reference values. For example, the processing device 120A may determine the development stage of the contrast agent corresponding to the current reconstruction angle range, and determine the reference value of the development stage as the reference value corresponding to the current reconstruction angle range. Since the rotation speed of the imaging device and the duration of each development stage are known, the rotation angle range corresponding to each development stage can be determined. The processing device 120A may determine the development stage corresponding to the current reconstruction angle range by determining which rotation angle range covers the current reconstruction angle range. If there are two or more rotation angle ranges of two development stages that cover the current reconstruction angle range, the development stage with the smallest reference value may be determined as the development stage corresponding to the reconstruction angle range.

After determining the reference value corresponding to the current reconstruction angle range is determined, the processing device 120A may determine a step value of the current reconstruction angle range, and determine the current reconstruction angle range by adding the step value to the previous reconstruction angle range.

FIG. 5 is a flowchart illustrating an exemplary process for generating one or more predicted reconstruction images according to some embodiments of the present disclosure. In some embodiments, one or more operations of the process 500 may be performed to achieve at least part of operation 340 as described in connection with FIG. 3.

In 510, the processing device 120A (e.g., the prediction module 240) may input the plurality of reconstruction images into the first trained image prediction model.

In some embodiments, the first trained image prediction model may be stored in a storage device in advance. The first trained image prediction model may be generated by the processing device 120B or another computing device by training a first preliminary model using a plurality of first training samples. For example, the processing device 120B (e.g., the obtaining module 250) may obtain the first training samples. Each of the first training samples may include a plurality of sample reconstruction images and one or more sample predicted reconstruction images. The sample reconstruction images and the one or more sample predicted reconstruction images may be generated based on sample scan data collected in a sample scan. Each of the one or more sample predicted reconstruction images may correspond to a reconstruction angle range that is between a reconstruction angle range of a sample reconstruction image in front of the sample predicted reconstruction image and a reconstruction angle range of a sample reconstruction image in the back of the sample predicted reconstruction image. In some embodiments, the sample reconstruction images and the one or more sample predicted reconstruction images may be a series of sample reconstruction images collected in a sample scan arranged according to the acquisition time, and the sample reconstruction images may be odd sample reconstruction images in the image series and the one or more sample predicted reconstruction images may be even sample reconstruction images in the image series. In some embodiments, the temporal resolution of the image series of each of the first training samples may fulfill a requirement of the temporal resolution and may be selected manually. In some embodiments, a first training sample may be generated by injecting the contrast agent into a sample subject more than once. In some embodiments, a first training sample may be generated by injecting the contrast agent into a sample subject at one time.

In some embodiments, the first trained image prediction model may be a machine learning model, such as a neural network model. For example, the first trained image prediction model may include a Fully Convolutional Network (FCN) model, a V-net model, a U-net model, an Alex network (AlexNet) model, a ResUNet model, a VB-net model, a Visual Geometry Group network (VGGNet) model, or the like, or any combination thereof.

In some embodiments, the processing device 120B (e.g., the training module 260) may be configured to generate the first trained image prediction model by training the first preliminary model based on the first training samples. In model training, the sample reconstruction images may be used as an input, and the one or more sample predicted reconstruction images may be used as the training ground truth, and the first trained image prediction model may be generated by iteratively updating the first preliminary model so that a terminal condition is satisfied (e.g., the difference between the model output of the first preliminary model and the training ground truth is minimized (e.g., smaller than a threshold)).

In 520, the processing device 120A (e.g., the prediction module 240) may generate the one or more predicted reconstruction images.

In some embodiments, the first trained image prediction model may output the one or more predicted reconstruction images.

Each of the one or more predicted reconstruction images may correspond to a reconstruction angle range that is between adjacent reconstruction angle ranges of the reconstruction images. The one or more predicted reconstruction images, combined with the reconstruction images, may enhance the temporal resolution of the reconstruction images.

FIG. 6 is a flowchart illustrating an exemplary process for generating a predicted reconstruction image according to some embodiments of the present disclosure. In some embodiments, one or more operations of the process 600 may be performed to achieve at least part of operation 340 as described in connection with FIG. 3.

In 610, the processing device 120A (e.g., the prediction module 240) may input one pair of adjacent reconstruction images into the second trained image prediction model.

In some embodiments, the processing device 120A may obtain at least one pair of adjacent reconstruction images among the plurality of reconstruction images and the second trained image prediction model. For each of the at least one pair of adjacent reconstruction images, the processing device 120A may input the pair of adjacent reconstruction images into the second trained image prediction model. In some embodiments, the second trained image prediction model may be stored in a storage device in advance. The second trained image prediction model may be generated by the processing device 120B or another computing device by training a second preliminary model using a plurality of second training samples. For example, the processing device 120B (e.g., the obtaining module 250) may obtain the plurality of second training samples.

Each of the plurality of second training samples may include a pair of sample adjacent reconstruction images and a sample predicted reconstruction image. The pair of sample adjacent reconstruction images and the sample predicted reconstruction image may be generated based on sample scan data collected in a sample scan. The sample predicted reconstruction image may correspond to a reconstruction angle range between two reconstruction angle ranges of the sample adjacent reconstruction images. The images in each of the plurality of second training samples may fulfill a requirement of the temporal resolution and may be selected manually. In some embodiments, the pair of sample adjacent reconstruction images may be two adjacent odd sample reconstruction images in the image series as described in connection with FIG. 5, and the sample predicted reconstruction image may be an even sample reconstruction image between the two adjacent odd sample reconstruction images.

In some embodiments, the second trained image prediction model may be a machine learning model, such as a neural network model. For example, the second trained image prediction model may include a Fully Convolutional Network (FCN) model, a V-net model, a U-net model, an Alex network (AlexNet) model, a ResUNet model, a VB-net model, a Visual Geometry Group network (VGGNet) model, or the like, or any combination thereof . . .

In some embodiments, the processing device 120B (e.g., the training module 260) may be configured to generate the second trained image prediction model by training the second preliminary model based on the plurality of second training samples. In model training, the pair of sample reconstruction images may be used as an input, and the sample predicted reconstruction image may be used as the training ground truth, and the second trained image prediction model may be generated by iteratively updating the second preliminary model so that a terminal condition is satisfied (e.g., the difference between model output of the second preliminary model and the training ground truth is minimized (e.g., smaller than a threshold)).

In 620, the processing device 120A (e.g., the prediction module 240) may generate the predicted reconstruction image.

In some embodiments, for each of the at least one pair of adjacent reconstruction images among the plurality of reconstruction images, the processing device 120A (e.g., the prediction module 240) may generate a corresponding predicted reconstruction image. In some embodiments, the second trained image prediction model may output at least one predicted reconstruction image corresponding to the at least one pair of adjacent reconstruction images.

The predicted reconstruction image may correspond to a reconstruction angle range that is between reconstruction angle ranges of the pair of adjacent reconstruction images. The predicted reconstruction image, combined with the pair of adjacent reconstruction images, may enhance the temporal resolution of the pair of adjacent reconstruction images.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer-readable media having computer-readable program code embodied thereon.

A non-transitory computer-readable signal medium may include a propagated data signal with computer readable program code embodied therein, for example, in baseband or as part of a carrier wave. Such a propagated signal may take any of a variety of forms, including electromagnetic, optical, or the like, or any suitable combination thereof. A computer-readable signal medium may be any computer-readable medium that is not a computer-readable storage medium and that may communicate, propagate, or transport a program for use by or in connection with an instruction execution system, apparatus, or device. Program code embodied on a computer-readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object-oriented programming language such as Java, Scala, Smalltalk, Eiffel, JADE, Emerald, C++, C#, VB. NET, Python or the like, conventional procedural programming languages, such as the “C” programming language, Visual Basic, Fortran, Perl, COBOL, PHP, ABAP, dynamic programming languages such as Python, Ruby, and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof to streamline the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities, properties, and so forth, used to describe and claim certain embodiments of the application are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method for medical image reconstruction, comprising:
determining a target temporal resolution of a target scan on a target subject within a scanning angle range;
determining, based on the target temporal resolution, a plurality of reconstruction angle ranges each of which being within the scanning angle range;
obtaining scan data acquired by causing an imaging device to perform the target scan on the target subject within the scanning angle range;
generating a plurality of reconstruction images corresponding to the plurality of reconstruction angle ranges based on the scan data.

2. The method of claim 1, wherein
at least one overlapping angle range exists between at least one pair of adjacent reconstruction angle ranges of the plurality of reconstruction angle ranges, one overlapping angle range corresponding to one of the at least one pair of adjacent reconstruction angle ranges.

3. The method of claim 1,
the obtaining scan data further including:
obtaining real-time scan data collected by the imaging device during the target scan; and
the generating a plurality of reconstruction images corresponding to the plurality of reconstruction angle ranges based on the scan data further including:
for each of the plurality of reconstruction images, starting image reconstruction based on the real-time scan data after the imaging device rotates to a starting angle of the corresponding reconstruction angle range.

4. The method of claim 1, wherein the target scan is a perfusion scan, and the determining a target temporal resolution includes:
obtaining reference information relating to a contrast agent used in the perfusion scan, the reference information including at least one of a contrast agent concentration of the contrast agent, an injection volume, an injection speed, a development stage of the contrast agent, or accuracy requirement of perfusion parameters; and
determining the target temporal resolution based on the reference information.

5. The method of claim 1, the determining a plurality of reconstruction angle ranges including:
obtaining a rotation speed of the imaging device during the target scan;
determining a reference value of a step based on the rotation speed and the target temporal resolution, the step being an angle interval between adjacent reconstruction angle ranges among the plurality of reconstruction angle ranges; and
determining the plurality of reconstruction angle ranges based on the reference value.

6. The method of claim 5, wherein the reference value of the step includes a plurality of reference values, and the angle interval between adjacent reconstruction angle ranges among the plurality of reconstruction angle ranges is changing.

7. The method of claim 6, wherein the target scan is a perfusion scan,
the target temporal resolution includes a plurality of target temporal resolutions corresponding to a plurality of development stages of a contrast agent used in the perfusion scan, and
each of the plurality of reference values is determined based on one of the plurality of target temporal resolutions and the rotation speed.

8. The method of claim 1, further comprising:
generating one or more predicted reconstruction images based on the plurality of reconstruction images, each of the one or more predicted reconstruction images corresponding to a reconstruction angle range that is between two adjacent reconstruction angle ranges among the plurality of reconstruction angle ranges.

9. The method of claim 8, wherein the generating the one or more predicted reconstruction images based on the plurality of reconstruction images includes:
obtaining a first trained image prediction model; and
generating the one or more predicted reconstruction images by inputting the plurality of reconstruction images into the first trained image prediction model.

10. The method of claim 8, wherein the generating the one or more predicted reconstruction images based on the plurality of reconstruction images includes:
obtaining at least one pair of adjacent reconstruction images among the plurality of reconstruction images;
obtaining a second trained image prediction model; and
for each of the at least one pair of adjacent reconstruction images, generating a predicted reconstruction image by inputting the pair of adjacent reconstruction images into the second trained image prediction model.

11. The method of claim 1, wherein a temporal resolution of the plurality of reconstruction images is equal to or higher than the target temporal resolution.

12. A system for medical image reconstruction, comprising:
at least one storage device including a set of instructions; and
at least one processor configured to communicate with the at least one storage device, wherein, when the instructions are executed, the at least one processor is configured to direct the system to perform operations, including:
determining a target temporal resolution of a target scan on a target subject within a scanning angle range;
determining, based on the target temporal resolution, a plurality of reconstruction angle ranges each of which being within the scanning angle range;
obtaining scan data acquired by causing an imaging device to perform the target scan on the target subject within the scanning angle range;
generating a plurality of reconstruction images corresponding to the plurality of reconstruction angle ranges based on the scan data.

13. The system of claim 12, wherein
at least one overlapping angle range exists between at least one pair of adjacent reconstruction angle ranges of the plurality of reconstruction angle ranges, one overlapping angle range corresponding to one of the at least one pair of adjacent reconstruction angle ranges.

14. (canceled)

15. The system of claim 12, wherein the target scan is a perfusion scan, and the determining a target temporal resolution includes:
obtaining reference information relating to a contrast agent used in the perfusion scan, the reference information including at least one of a contrast agent concentration of the contrast agent, an injection volume, an injection speed, a development stage of the contrast agent, or accuracy requirement of perfusion parameters; and
determining the target temporal resolution based on the reference information.

16. The system of claim 12, the determining a plurality of reconstruction angle ranges including:
obtaining a rotation speed of the imaging device during the target scan;
determining a reference value of a step based on the rotation speed and the target temporal resolution, the step being an angle interval between adjacent reconstruction angle ranges among the plurality of reconstruction angle ranges; and
determining the plurality of reconstruction angle ranges based on the reference value.

17. The system of claim 16, wherein the reference value of the step includes a plurality of reference values, and the angle interval between adjacent reconstruction angle ranges among the plurality of reconstruction angle ranges is changing; and
wherein the target scan is a perfusion scan,
the target temporal resolution includes a plurality of target temporal resolutions corresponding to a plurality of development stages of a contrast agent used in the perfusion scan, and
each of the plurality of reference values is determined based on one of the plurality of target temporal resolutions and the rotation speed.

18. The system of claim 12, wherein the at least one processor is configured to direct the system to perform further operations including:
generating one or more predicted reconstruction images based on the plurality of reconstruction images, each of the one or more predicted reconstruction images corresponding to a reconstruction angle range that is between two adjacent reconstruction angle ranges among the plurality of reconstruction angle ranges.

19. The system of claim 18, wherein the generating the one or more predicted reconstruction images based on the plurality of reconstruction images includes:
obtaining a first trained image prediction model; and
generating the one or more predicted reconstruction images by inputting the plurality of reconstruction images into the first trained image prediction model.

20. The system of claim 18, wherein the generating the one or more predicted reconstruction images based on the plurality of reconstruction images includes:
obtaining at least one pair of adjacent reconstruction images among the plurality of reconstruction images;
obtaining a second trained image prediction model; and
for each of the at least one pair of adjacent reconstruction images, generating a predicted reconstruction image by inputting the pair of adjacent reconstruction images into the second trained image prediction model.

21. (canceled)

22. A computer-readable non-transitory storage medium storing computer instructions, and when a computer reads the computer instructions in the non-transitory storage medium, the computer executes a method for medical image reconstruction, comprising:
determining a target temporal resolution of a target scan on a target subject within a scanning angle range;
determining, based on the target temporal resolution, a plurality of reconstruction angle ranges each of which being within the scanning angle range;
obtaining scan data acquired by causing an imaging device to perform the target scan on the target subject within the scanning angle range;
generating a plurality of reconstruction images corresponding to the plurality of reconstruction angle ranges based on the scan data.
